(19) United States
(12) Patent Application Publication
(10) Pub. No.: US 2021/0375485 A1
(43) Pub. Date: Dec. 2, 2021

(54) APPLICATION FOR TRACKING INFECTIOUS DISEASE

(71) Applicant: OptimDosing, LLC, Farmington Hills, MI (US)

(72) Inventors: Kenneth I. KOHN, West Bloomfield, MI (US); David INWALD, Berkley, MI (US); Caitlin Joline BROWN, Ashburn, VA (US)

(21) Appl. No.: 17/333,999

(22) Filed: May 28, 2021

Related U.S. Application Data
(60) Provisional application No. 63/031,173, filed on May 28, 2020.

(51) Int. Cl. G16H 50/80; G16H 10/20; G16H 10/60; G16H 50/30; G16H 50/20; G16H 80/00; G16H 40/67; G06N 20/10

(52) U.S. Cl. CPC G16H 50/80; G16H 10/20; G16H 10/60; G06N 20/10; G16H 50/20; G16H 80/00; G16H 40/67; G16H 50/30

(57) ABSTRACT

An application for tracking infectious disease including an input module for inputting variables from a user in electronic communication with an output variable module, an analysis module for analyzing input variables and output variables, and an output module for presenting results to the user. A method of tracking infectious disease, by a user inputting data about symptoms of infectious disease and user defined metrics in an application, performing an analysis on the data, and outputting a result from the data tracking symptom progression, tracking geolocation of symptoms and outbreaks, and tracking trends among individuals without symptoms and individuals with different diseases. A method of monitoring the health of employees and students.

### BACKGROUND OF THE INVENTION

### 1. Technical Field

The present invention relates to methods of tracking daily or periodic activity and symptoms of diseases and mental health issues. More specifically, the present invention relates to methods of tracking infectious disease.

### 2. Background Art

Infectious diseases are diseases caused by pathogenic microorganisms such as bacteria, viruses, parasites, or fungi that can be spread from person to person, either directly or indirectly.

Coronavirus Disease 2019 (COVID-19) is a severe acute respiratory syndrome (SARS) coronavirus 2 that originated in 2019 in Wuhan, China, and has quickly spread around the world. The viral infection is spread from person to person by respiratory droplets. Symptoms include fever, cough, shortness of breath, fatigue, muscle or body aches, headache, new loss of taste or smell, sore throat, congestion or runny nose, nausea or vomiting, and diarrhea and it can be very similar to influenza. While tests are available to identify COVID-19, they are generally not being used on people with milder symptoms due to the cost of tests or lack of available of tests. Individuals who have been identified as having the virus need to quarantine themselves. It would be advantageous to track individual's symptoms if they are not feeling well as well as tracking habits of quarantined individuals to ensure compliance. There is emerging evidence that COVID will be an annual, seasonal infection. Thus, long-term tracking will be important for the foreseeable future. Likewise, the tracking application can be applied to other infectious diseases, on the same global pandemic scale as COVID-19 or for more localized outbreaks, e.g., ebola, measles, etc., or on a localized scale, e.g., lice outbreak within an elementary school.

It would further be advantageous to be able to track individuals once they return to work environments. Businesses need to be aware of the health of their employees so that their employees can safely return to the workplace, and so that if an individual starts experiencing any symptoms of COVID-19, the employee can be told to stay home and monitor themselves for any worsening conditions as well as notify other employees that they may be at risk. Nearly two-thirds of people surveyed are experiencing some anxiety due to COVID-19. Further contributing to the stress is the confusion caused by the lack of coordinated guidelines for the health and safety of workplaces. Instead, local governments are responsible for issuing their own standards, leading to inconsistency and, sometimes, inadequacy. As such, proactive organizations and corporate entities impose their own, often stricter criteria to determine when employees are fit to return to the workplace. Screening software can bring peace of mind to employers and employees by helping to ensure coworkers all meet the same criteria when returning to work.

Therefore, there remains a need for a method of tracking infectious disease, a need for predicting adverse events and individual susceptibility so that they can be avoided or treated in time, and a need for monitoring employees with potential symptoms.

### SUMMARY OF THE INVENTION

The present invention provides for an application for tracking infectious disease including an input module for inputting variables from a user in electronic communication with an output variable module, an analysis module for analyzing input variables and output variables, and an output module for presenting results to the user.

The present invention provides for a method of tracking infectious disease, by a user inputting data about symptoms of infectious disease and user defined metrics in an application, performing an analysis on the data, and outputting a result from the data tracking symptom progression, tracking geolocation of symptoms and outbreaks, and tracking trends among individuals without symptoms and individuals with different diseases.

The present invention also provides for a method of monitoring the health of employees or students, by an employee or student inputting data about symptoms of infectious disease and user defined metrics in an application, performing an analysis on the data, outputting a result from the data tracking symptom progression, alerting an employer/school about the status of the employee's or student's symptoms, and indicating either that the employee or student should be sent home or continue to work.

### DESCRIPTION OF THE DRAWINGS

Other advantages of the present invention are readily appreciated as the same becomes better understood by reference to the following detailed description when considered in connection with the accompanying drawings wherein:

FIG. 1 is a diagram of the flow of information in the application and method;

FIG. 2 is a macro-level systems design of the present invention;

FIG. 3 is a diagram of the flow of information in the application when used for workplace readiness assessment;

FIG. 4 is a screenshot view of creating a workplace;

FIG. 5 is a screenshot view of a workplace dashboard;

FIG. 6A is a screenshot view of adding an individual employee, and FIG. 6B is a screenshot view of adding employees in bulk;

FIG. 7 is a screenshot view of employee sign up;

FIG. 8 is a screenshot view of an employee questionnaire;

FIG. 9 is a screenshot view of an employee dashboard;

FIG. 10 is a screenshot view of a telehealth interview; and

FIG. 11 is a heatmap of use of the application of the present invention.

### DETAILED DESCRIPTION OF THE INVENTION

The present invention generally provides for a user friendly application (shown at 10 in the FIGURES) and method of use that quickly captures daily activities, intake, and symptoms of users with diseases and mental health issues to find otherwise hidden patterns in order to determine symptom triggers and effects on their body, and especially symptom progression in infectious disease. The information can be input by the user answering preset questions. The types and quantity of data entered by a user varies depending on the workplace and condition, but the average user is able to complete daily interactions with the application in less than one minute. In addition to user-direct inputs, the information can be gathered from existing and newly developed outside monitoring devices. These monitoring devices can measure cardiac, circulatory or other physical properties of the user over time. The information gathered is analyzed over time along with patient gathered data gathered over time. This information enables users to make modifications to their lifestyle to ultimately feel better. The information can also be used to predict an adverse event happening at a later time point so that the user can either prevent the adverse event from happening with lifestyle changes or receive treatment to prevent the adverse event.

The term “application” as used herein refers to a computer software application, otherwise known as an “app”, that is run and operated on a mobile device, such as, but not limited to, smart phones (IPHONE® (Apple, Inc.), ANDROID™ devices (Google, Inc.), WINDOWS® devices (Microsoft)), mp3 players (IPOD TOUCH® (Apple, Inc.)), or tablet computers (IPAD® (Apple, Inc.)), especially ones utilizing a touch screen. The application can also be web based and run on a computer or laptop. The application 10 includes any necessary user interface or display and storage components to display the application and store the algorithm running it.

“Diseases and mental health issues” as used herein can include diseases such as digestive disorders or migraines, and mental health issues such as anxiety attacks, or suicidal thoughts, among others. The diseases and mental health issues are preferably ones that are affected by outside triggers such as diet and lifestyle or environment.

“Infectious disease” as used herein can include an viral, protozoan, or bacterial disease such as most preferably influenza, measles, or COVID-19, or any of AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68 fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, or zika virus.

“Trigger” as used herein, refers to an event or situation that causes or provokes a disease or condition to happen.

“Adverse event” as used herein, refers to any medical occurrence that is undesired in a user. Examples can include, but are not limited to, headaches, nausea, heart attacks, seizures, allergic reactions, hemorrhages, tissue damage, or any other damage to the body. Adverse events can cause disability, permanent damage, or even death.

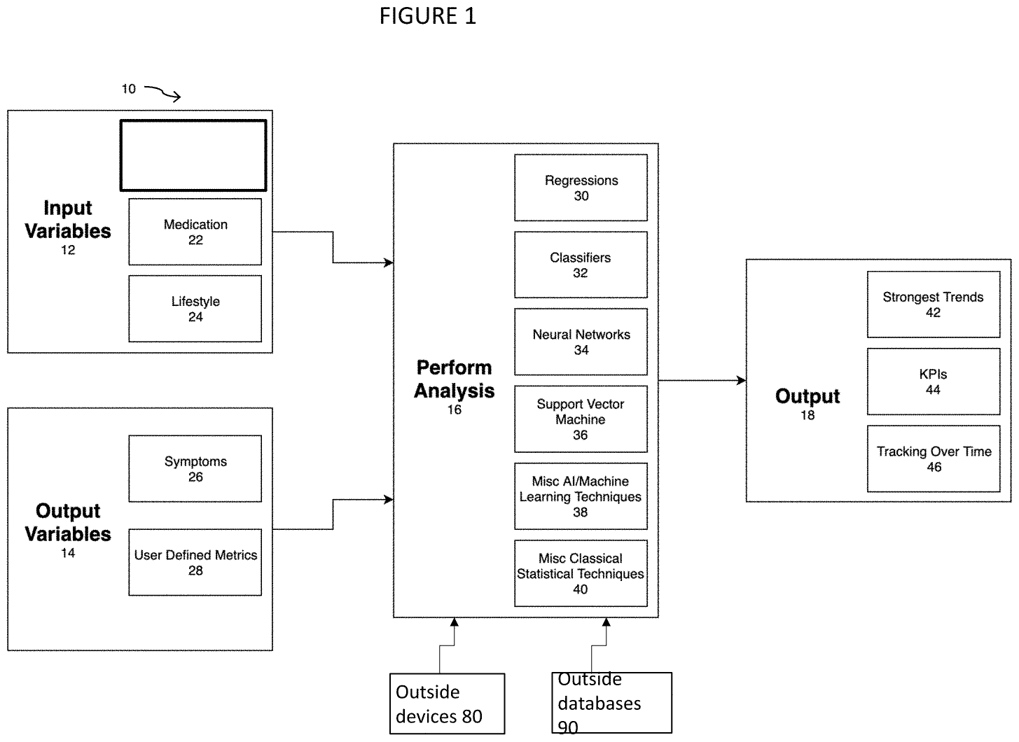

As generally shown in FIG. 1, the application 10 includes an input module 12 for inputting variables from a user in electronic communication with an output variable module 14, an analysis module 16 that analyzes data from the input variables and output variables, and an output module 18 for presenting results to the user. Each of these modules can be run by algorithms stored on non-transitory computer readable media.

The input module 12 can be used to keep a daily log of users' lifestyle and symptoms. The questions are kept very simple so that a user can complete them in 1-2 minutes. The input module 12 can include a medication question module 22 and a lifestyle question module 24. Questions presented can be answered on a continuous or nominal scale. Input can also be gathered from various medical devices, such as portable monitoring systems, further described below. Accordingly, cardio, vascular, and neuro information can be input.

With the medication question module 22, the user can input any medication they are taking, including vitamins and supplements, with dosing schedules and amounts.

With the lifestyle question module 24, questions can be presented to the user such as (with available answer choices in brackets):

How many hours of sleep did you get last night? [0 to 12+, on 0.5 intervals]

Did you work out today? [yes or no]

Did you take time to relax today [yes or no]?

How stressed did you feel today? [0 to 5 scale]

The lifestyle question module 24 can also generally include questions regarding anxiety and mental health

The output variable module 14 can include a symptom question module 26 and a user defined metrics question module 28.

With the symptom question module 26, questions can be presented to the user such as (with available answer choices in brackets):

How much pain were you in today? [0 to 5 scale]

How many bowel movements did you have today? [0 to 10+, or on Bristol scale]

How many times did you pass blood? [0 to 10+]

Did you have a headache today? [yes or no]

The symptom question module 26 can further include questions related to infectious diseases, such as:

Do you have a cough?

[No]

[Yes→Is it a dry cough or wet cough? (a wet or productive cough means there is fluid in your airways, a dry cough means there is no fluid in your airways), select from wet cough, dry cough, or not sure]

Do you have shortness of breath? [yes or no]

What is your temperature? [Enter #]

[Not sure]→Do you think you have a fever? [yes or no]

Have you had any digestive issues? (e.g., diarrhea, vomiting, etc.)

[No]

[Yes]→

Have you had diarrhea? [yes or no]

Have you felt nauseous? [yes or no]

Have you vomited? [yes or no]

Have you had other abdominal discomfort? [yes or no]

Are you experiencing any of the following? [body aches, chills, fatigue, headache, postnasal drip, runny nose, sinus congestion, skin rash, sneezing, sore throat, swollen glands, watery eyes, loss of smell, loss of taste]

Have you been in contact with anyone with a positive COVID-19 diagnosis?

[No]

[Yes] →When were you in contact?

Where were you in contact?

On a scale of 1 (not at all anxious) to 10 (extremely anxious), how anxious did you feel today?

Have you limited your daily activities?

[No]

[Yes] →Have you self-quarantined? [Yes or No]

With the user defined metrics question module 28, the user can design any other relevant questions and answers that could relate to their disease or condition that can be added to the application 10 to include in an analysis, such as alcohol intake, traveling, or preexisting chronic conditions.

All the data collected from the input module 12 and the output variable module 14 is sent to the analysis module 16. The analysis module 16 can include regressions 30, classifiers 32, neural networks 34, support vector machine 36, miscellaneous Al/machine learning techniques 38, and/or miscellaneous classical statistical techniques 40 in performing the analysis of the data.

In general, the analysis module 16 uses the data to find patterns between user responses and their potential disease state. By estimating multiple regressions 30 on time lagged variables, the application 10 can find patterns otherwise unnoticeable. With just one week of data, connections can be identified between user responses and the state of infectious disease.

The disease state or otherwise pass/fail criteria can be used as the dependent variable in a series of regressions 30. The failing criteria variables include both same day, as entered values and time lagged, such that the first row of data is deleted out to four days later, and might include social related questions such as indications for recent travel, hosting travelling guests, etc. The symptom data measured are used as the independent, or predictor, variables. Linear regressions 30 are then estimated to determine which independent variables cause an increase in the symptoms, or dependent variables. The specific mechanisms are as follows. Users input their responses, each on a continuous or nominal (from Likert-type items) scales. The response variables include both same day, as entered values, and time lagged, such that the first row of data is deleted out to four days later. The responses measured are used as the independent, or predictor, variables. Linear regressions are then estimated to determine which independent variables cause an increase in the likelihood of a positive disease state or failed criteria. Specifically, the response variables are then used as the dependent variables in a series of linear, ordinary least regressions. Within the first month of use, regressions are estimated for each type of failed criteria. Each regression coefficient with alpha <0.2 is flagged to users as a potential factor contributing to their symptoms. After users have inputted a full month of data, one master regression is estimated for each symptom outcome, combining the predictor variables, thereby allowing the relative impact across categories to be determined. With the full month of data, the significance level drops to alpha <0.4.

Linear regressions 30 test the null hypothesis that the relationship between the independent variable(s) and dependent variable is 0. Unlike traditional data analysis, which requires a 5% alpha level to claim significance, the threshold for flagging potential lifestyle problems is lower. Specifically, the 5% standard level translates to a 95% likelihood that an effect is not due to chance, thereby rejecting the null hypothesis that the relationship is 0. Further, the system can time lag outcome variables to capture the impact of day-to-day life on symptoms the same day, the next day, and the day after that. These regressions 30 serve as the steps in an algorithm.

While regressions 30 can be preferred, other methods of analysis can be used. Classifiers 32 are a broad use of artificial intelligence and machine learning that determine the relationship between input variables and output variables are categories. In the case of the present invention, it can be classified whether or not a specific user's data classifies as fitting the profile of effective lifestyle changes to help improve symptoms.

Miscellaneous Machine Learning Techniques 38 can include other common Al techniques and combination of techniques.

Miscellaneous Classical Statistical Techniques 40 can include looking at distributions of data, means, deviations, tracking over time, etc. These techniques are commonly used as a part of feature extraction (to supplement the user-submitted data when running the models).

The present invention also enables rule-based messaging where application administrators can set predetermined pass/fail criteria based on their specific set of questions prompted to their users. Any single specific response, or combination of responses with Boolean or continuous operators can determine a failed response. Upon identifying a set of responses as “failed” the system is capable of alerting designated contacts of the failed result. As an example, the system might want to alert the user to a custom email or text messaging providing them further instruction/direction, as well as notify that individual's Human Resources representative that they failed and must meet further criteria in order to be granted access.

The present invention enables targeted, stratified shutdown, e.g., floors, classrooms, based on exposure and likelihood of infection. One limitation of GPS and Bluetooth contact tracing efforts is apparent with multi-level workplaces where applications cannot distinguish between people at different floors in the same building. The present invention allows arbitrary groupings of individuals within an organization, such that buildings, floors, grade-levels, can be separated for the purpose of analysis and response planning. Subgroup classification enables targeted quarantines, meaning entire workplaces might not need to close if the group can be contained.

Further, the present invention enables informed predictions to be made for infection risk based on cluster probabilities. There are several methodologies that enable this.

First, nearest neighbor algorithms can also be performed once a large enough group of users are using the application 10. A multi-dimensional nearest neighbor algorithm is used to find those individuals from existing sets, i.e., a K-Nearest Neighbor (KNN) algorithm. The KNN algorithm is a clustering algorithm and acts as a non-parametric untrained classifier that evaluates the overall similarity between two users based on the degree of differences across multiple features. The flexibility of such an algorithm allows consideration of many parameters when searching for pertinent context data. Weights on certain factors can vary depending on the type of symptom. These similar user profiles are grouped into subsets to look for trends that can be used to optimize the suggestions for the user. While the KNN algorithm can be preferred, other clustering algorithms can also be used, such as, but not limited to, K-Means, Affinity Propagation, Mean Shift, Spectral Clustering, Support Vector Machines. One advantage of KNN over other techniques is that it is easily scalable across many dimensions. Further, from case-to-case the differing dimensions and weights are easily included.

The purpose of the KNN algorithm is to find users most similar to the present user. Once identified, the “neighboring” user data are used to evaluate the present user. To make the identification, the differences in each parameter comprising the user data structure are evaluated. While most commonly used with continuous values (weight, age, LDL level, etc.), the algorithm can be used with discrete values as well (race/ethnicity, familial history, presence of certain symptoms, DNA information, etc.). The differences across each parameter are combined using a weighting scheme such that a normalized ‘distance’ is produced representing an overall difference metric between two users. The distance calculation between two users is achieved using a regression-type KNN algorithm. Key to the regression evaluations is the Mahalanobis distance. The Mahalanobis distance evaluates to a Euclidian distance since the covariance matrix is always the identity matrix, i.e., one parameter in this case is never to be compared independently with another parameter. The benefit of adapting the Mahalanobis distance instead of using pure Euclidian distance is that Mahalanobis distance includes the measurement of the number of deviations away from the norm. While the actual standard deviation is not always ideal, an equivalent term is used.

If the present user P1 has a set of parameters where P₁={μ_{1P1}, μ_{2P1}, μ_{3P1}, . . . μ_{NP1}}P₁={μ_{1P1}, μ_{2P1}, μ_{3P1}, . . . μ_{NP1}} and an arbitrary user, P_{β}P_{β}, where P_{β}={μ_{1Pβ}, μ_{2Pβ}, μ_{3Pβ}, . . . μ_{NPβ}}P_{β}={μ_{1Pβ}, μ_{2Pβ}, μ_{3Pβ}, . . . μ_{NPβ}}, then the distance DD between the two users is:

D₁(P₁,P_{β})=\sqrt{Σᵢ₌₁^{N}(μ_{iP1}−μ_{iPβ})²}

Several adaptations are needed to the above generalized equation. Mainly, handling a weighting schema. Most simply, a set of weights, W, should be created with each parameter in P being assigned a weight. Weights can be applied using any technique. Shown below is an intuitive 1-10 linear weighting schema. If W={ρ₁, ρ₂, ρ₃, . . . ρ_{N}} W={ρ₁, ρ₂, ρ₃, . . . ρ_{N}}, then the distance, DD, can be evaluated by:

D₂(P₁,P_{β})=\sqrt{Σᵢ₌₁^{N}ρᵢ(μ_{iP1}−μ_{iPβ})²}

In the above examples for D₁D₁ and D₂D₂ continuous values are used for μ_{N}μ_{N}. In this application, continuous values can be integers or rational numbers. Discrete values must be handled in a special manner. Since there is no intuitive value for the difference between two ethnicities, one must be manually supplied in a lookup table. Algorithmically, parameters with continuous values should be summated using the squared difference while parameters with continuous values are summated manually. The same W={ρ₁, ρ₂, ρ₃, . . . ρ_{N}} W={ρ₁, ρ₂, ρ₃, . . . ρ_{N}} weighting schema applies to discrete parameters as well. Further, the ultimate output can be either a continuous probability score, which can be converted to a binary function once a threshold for risk is established, such that above the probability cutoff flags the user and below the cutoff does not.

The threshold for evaluating whether or not another user is sufficiently similar to the present user is situational. The ideal number of similar subjects is to be optimized on a case-to-case basis when there exists sufficient training data.

KNN algorithms have been used before. For example, U.S. Pat. No. 10,123,748 (IBM) discloses a Patient Risk Analysis method that uses KNN to find similar patients. U.S. Pat. No. 7,730,063 discloses a personalized medicine method that also mentions KNN as a potential algorithm for finding similar patients. The present invention's ability to include continuous and discrete parameters as well as customized weights in the KCN differentiates over these prior art methods.

After the analysis, strongest trends 42, key performance indicators (KPIs) 44, and tracking over time 46 are sent to the output module 18 and displayed to the user. For example, predictor variables that meet a 60% or greater threshold are output to users with the output module 18 and flagged as potential causes of their symptoms or KPIs 44. Users are then encouraged to keep tracking to increase the predictive power. Predictor variables meeting a more stringent 90% threshold are flagged as likely causes, or strongest trends 42. Users are then encouraged to talk to their doctors to determine how they can improve their symptoms. Alternatively, the application 10 can be in communication with external databases and/or doctors/healthcare professionals that can suggests changes to improve their symptoms. Users can review statistics of the outputs by week, month, or year with tracking over time 46.

Second, time series is a system of data points organized by time. Time then becomes one of the key predictors of an outcome, by looking at autocorrelation, seasonality, and stationarity. Time series enable an understanding of how data vary over time and how changes in a given variable over time compare to changes in other variable over time. Risk of infection inherently changes over time, as level of contagiousness changes. With COVID-19, those infected tend to be contagious for two days prior to symptoms to 14 days after. The degree of contagiousness is still being investigated and can be a key output of this application.

Time series can follow several broad patterns: trends occur when there is an overtime increase or decrease in a data series; seasonal patterns occur when data over time are impacted by external changes at a fixed and known frequency, like time of the week, month, year, etc., and cycles occur when changes in data over time correlate with other, non-fixed external changes. In this application, trends can occur as medical prognosis generally improves or deteriorates. Seasonal patterns can be due to environmental factors that map the spread of COVID, like temperature and humidity. Time series analysis can enable the application to account for changes likelihood of infection over time, as well as changes in adverse symptom outcomes as they relate to related seasonal and cyclical changes in the seasons.

Neural Networks (NNs) 34 are another broad Al/machine learning technique that can be used to detect patterns in data. Previous use cases for neural networks include real-time translation, facial recognition, and music composition. Neural networks map inputs to outputs via a series of algorithms designed to loosely model the human brain. Specifically, each input is entered as a vector that makes up the left-side layer of a broader neural network. For this application, the inputs include exposure, demographics, and location. The right-side layer of a neural network is the output. In this application, the output includes all adverse symptom outcomes. Between the input and output layers is a hidden layer, which is a weighted sum of the values in the input layer that projects the outcome layer, thereby determining how the inputs work together to create the outputs. This hidden layer determines how symptoms, and user defined metrics work together to create symptom outputs.

Neural networks follow an iterative process between forward and backward propagation. In forward propagation, the weights in factors of the hidden layers are calculated to determine output layer prediction and error probability of that prediction. Backward propagation runs in the opposite direction, bringing higher error likelihood from the right output layer back into the hidden layers to adjust the weights. This in turn decreases the likelihood of error at the output layer. In this application, the hidden layers determine the weights for the different nutrition, medication, lifestyle, symptoms, pain, and user defined metrics to predict adverse symptom outcomes in the output layer. If the error of that prediction exceeds a certain level, back propagation returns to the hidden layers to adjust the weights and increase the probability that the adverse symptom prediction is accurate. Forward and backward propagation are iterative until the output, or adverse symptom event, is predicted with greater certainty.

Deep neural networks add additional hidden layers that aggregate and recombine data from the previous layer. The current application will use the additional layers of deep neural networks to cluster nutrition, medication, lifestyle, symptoms, pain, and user defined metrics together over time. Thus, clusters of behavior across time will more accurately predict adverse symptom outcomes. Deep learning networks use automatic feature extraction, enabling the machine to identify patterns without the need for human intervention, thereby mitigating bias. For the present invention, neural networks are one of the strategies used to identify trends in the data. NN models can be used for analyzing certain symptoms or broadly over the data set.

Support Vector Machines (SVMs) 36 can be used as part of the classification technique to identify certain features. SVMs are supervised learning models that rely on attempting regressions to evaluate which have the strongest fit with the data set. SVM assumes a binary outcome. In the case of this application: did the adverse symptom occur on a given day or not. SVM then makes a non-probabilistic binary linear classifier by plotting points in space. These points represent factors contributing to the likelihood of the outcome, i.e., symptoms, and user defined metrics. The bigger the gap between the clusters, the better the predictive power, as the potential binary outcomes sit relatively farther apart [KB4].

In most real world examples, however, the gap between one outcome versus the other is non-existent, with much overlap. This is likely the case with predicting adverse symptoms, as the predicting symptoms, and user defined metrics likely bleed together. To account for this, the application can use the Kernel Trick. Kernel functions compute the similarity between inputs according this formula, where x and y are input vectors, ϕ is a transformation function, and < > refers to the dot product:

K(x,y)=<ϕ(x),ϕ(y)>

If the dot product is small, the functions are different; if it is large, there is more overlap. The Kernel trick then looks for transformations in the boundaries between the x and y by plotting the functions in multi-dimensional space in order to keep a linear classifier. Because we expect overlap in the symptoms, and user defined metrics that predict whether or not an adverse symptom will occur, the Kernel trick will enable the combinations of factors to be plotted multi-dimensionally in order to define a natural linear divide between a symptom occurring versus not occurring. This in turn defines which symptoms, and user defined metrics and in which combination contribute to an adverse symptom outcome.

Random forest algorithms [KB5] are a method for classification and regression that creates a series of decision trees to predict the alignment of a given input to a given tree. Specifically, random forests look at the predictive power of the full system of factors to determine the underlying function, plus noise. Random forest classification starts with a decision tree, wherein an input is entered at the top of the tree and travels down each branch. In the case of this application, the input would be an adverse symptom outcome, with each branch being the range of answers on a given predictive factor or series of predictive factors. Each day of inputted data would be its own tree, with the input being adverse symptom outcome and the branches for each of the predictors tracked. Random forests look at the average across a series of such trees to make a stronger prediction of an adverse outcome. The larger the number of trees, the more accurate the ultimate forest prediction. In this application, each tree is a day of data and the more days collected, the more accurate the predictions. Random forest algorithms identify the most important features. Random forests will therefore enable this application to identify the most salient factors from the tracked symptoms, and user defined metrics. Random forests are also particularly adept at handling missing data, as is likely the case with user input daily logs. Random forest can help classify symptom groupings to better predict and manage symptoms.

With respect to infectious disease, the application 10 can output from the data tracking of symptom progression (both within individuals and within regions), tracking of anxiety over time (regionally and related to symptoms, exposure, and activity limiting), tracking geolocation of symptoms and outbreaks, and tracking trends among individuals without symptoms or individuals with different diseases (i.e. symptoms can be tracked not associated with a particular infectious disease such as COVID-19 but that go with cold, flu, allergies, etc.)

The application 10 can be in communication with databases, government facilities, or medical facilities regarding data collected. The data can be provided in anonymous or aggregate form or in identifiable form so that appropriate government and medical personnel can identify potential infectious individuals and their contacts to prevent further spread of disease.

The application 10 can also include any suitable alarms or notifications that can remind users to input data into the input module 12 or output variable module 14 at certain times of the day or daily. Such notifications can be pushed to the user's mobile devices such as a smart phone, smart watch, tablet, or desktop or laptop computer.

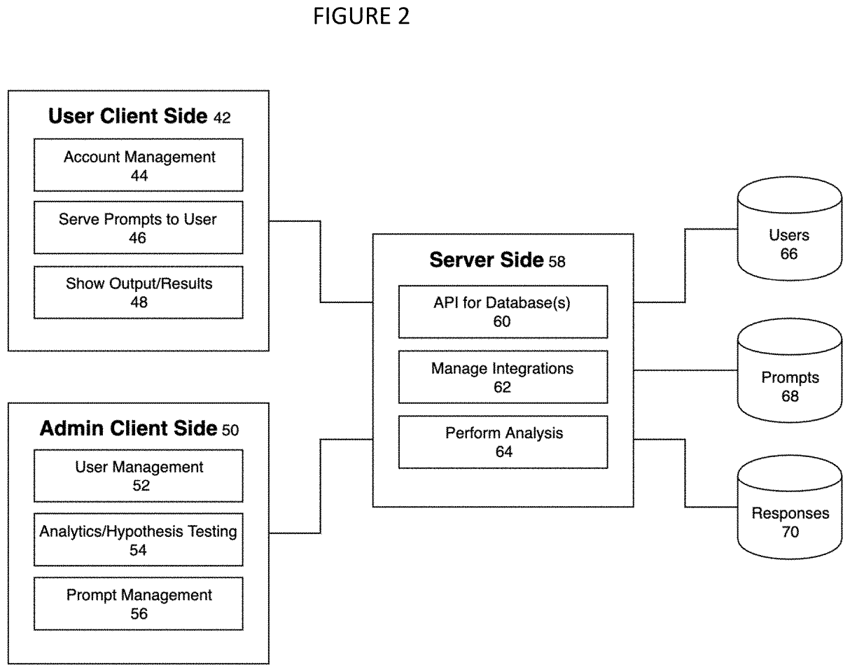

FIG. 2 shows a macro-level systems diagram. The User Client Side 42 includes the interactions the software has directly with the user. This includes interactions from native applications (iOS, Android), or web applications (accessed in a browser) and can include account management 44 (sign up, login, password management), serve prompts to user 46, and show output/results 48. The Admin Client Side 50 includes interactions “Admin” level users have access to, such as user management 52, analytics/hypothesis testing 54, and prompt management 56. The Server Side 58 outlines the major functions performed by the server. Application programming interface (API) for databases 60 can be performed. Integrations can be managed 62 including data from other health/nutrition trackers, fitness trackers, wearable devices, etc. Perform Analysis 64 refers to the breakdown represented in FIG. 1. Databases of users 66, prompts 68, and responses 70 can all be in electronic communication with the Server Side 58.

The present invention also provides for a method of tracking infectious disease, by a user inputting data about symptoms of infectious disease, and user defined metrics in an application, performing an analysis on the data, and outputting a result from the data tracking symptom progression, tracking anxiety over time, tracking geolocation of symptoms and outbreaks, and tracking trends among individuals without symptoms and individuals with different diseases. This method can be performed with the application 10 as described above.

As mentioned above, the application 10 can integrate and analyze data (at 62) from outside devices 80 that measure physiological properties of the user and are preferably wearable medical devices. These outside devices 80 can include, but are not limited to, general fitness trackers (FitBits®, Apple® Watch), heartbeat trackers, heart rate trackers, skin temperature trackers, respiratory rate trackers, body posture trackers, eyesight trackers, blood oxygen trackers, glucose level trackers, sleep trackers, body temperature trackers, and skin conductance trackers. Any other suitable physiological data can also be collected. The outside devices 80 can be separate devices or a combination in a single device. Preferably, the outside devices 80 generally provide electrophysiological monitoring.

Therefore, the present invention provides for a method of tracking infectious disease, by a user inputting data about infectious disease symptoms and user defined metrics in an application, integrating a user's data from outside devices, performing an analysis on the data, and outputting a result from the data tracking symptom progression, tracking anxiety over time, tracking geolocation of symptoms and outbreaks, and tracking trends among individuals without symptoms and individuals with different diseases.

The application 10 can also integrate and analyze data from outside databases 90, especially having clinical trial data, such as clinics, electronic medical records (EMRs), pharmaceutical companies, private databases, or CROs, further described in U.S. Provisional Patent Application No. 62/878,066. Nearest neighbors can be identified as described above and related study or trial data can be identified in the outside databases 90 to be analyzed. By analyzing additional outside data from the outside databases 90, the application can find others who have similar data as the user and predict an adverse event or triggers to an adverse event.

Therefore, the present invention provides for a method of tracking infectious disease, by a user inputting data about infectious disease symptoms and user defined metrics in an application, integrating data from outside databases, performing an analysis on the data, and outputting a result from the data tracking symptom progression, tracking anxiety over time, tracking geolocation of symptoms and outbreaks, and tracking trends among individuals without symptoms and individuals with different diseases.

The application 10 can be used by employers/school administration to monitor the health of their employees/students and can be used to determine when the employee/student can return to work/school or if the employee/student needs to stay home because of a risk of having an infectious disease (i.e., it can function as a workplace readiness assessment). In addition to providing a record of having met government requirements, the application 10 helps maximize safety and bring peace of mind to employers, their employees, and their customers as well as school administration and students. The application 10 implements policies based on parameters set by government standards or the employer/school, allowing employers/schools to screen employees/students, ensuring they are ready to return to work or school. Workplace readiness criteria is customized by the organization and can include screening for specific symptoms over a predetermined period of time, COVID-19, and antibody test results, etc. The application 10 also offers a method for telehealth to employers when face-to-face virtual interviews/exams with a clinician are required. Telehealth can be an effective means to increase the accuracy of the screening process, especially in marginal cases.

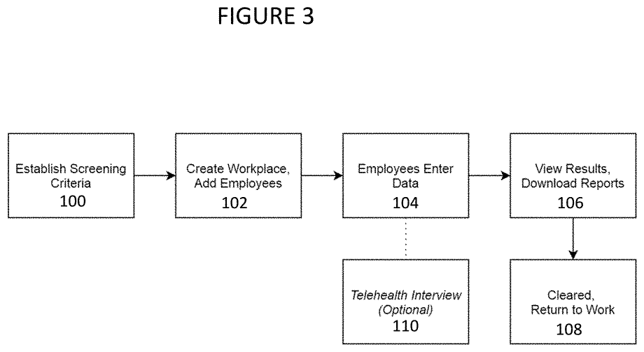

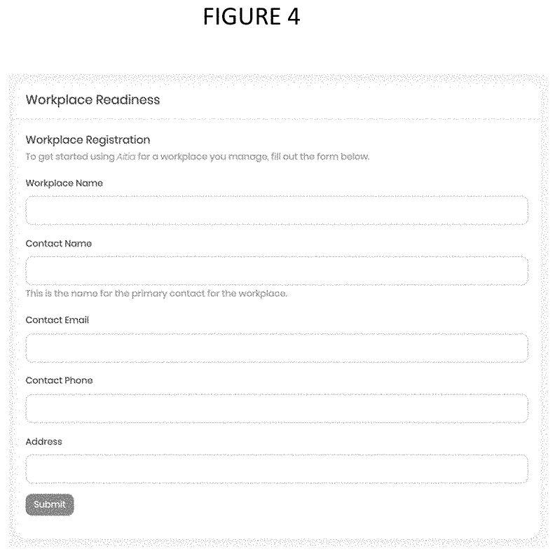

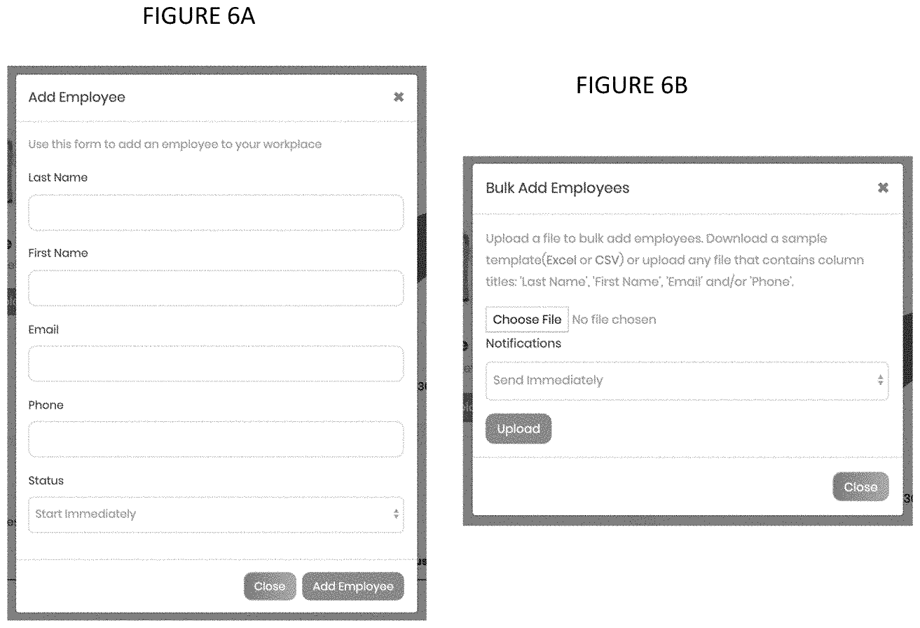

FIG. 3 shows a diagram of the flow of information in the application 10 in this method. Screening criteria are established 100, a workplace is created (also shown in FIG. 4) and employees are added 102 (also shown in FIGS. 6A and 6B), employees enter data 104, results can be viewed, and reports downloaded 106, and the employee can be cleared to return to work 108. A telehealth interview can be optional 110. This can also be performed with a school by adding 102 students.

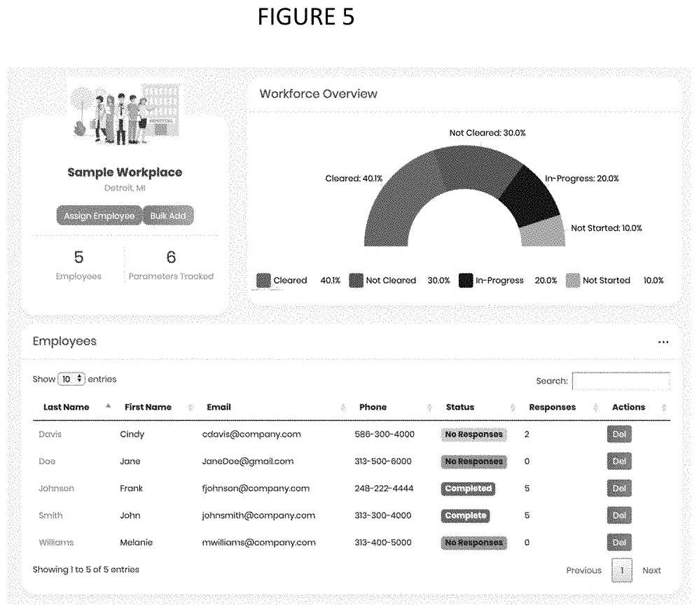

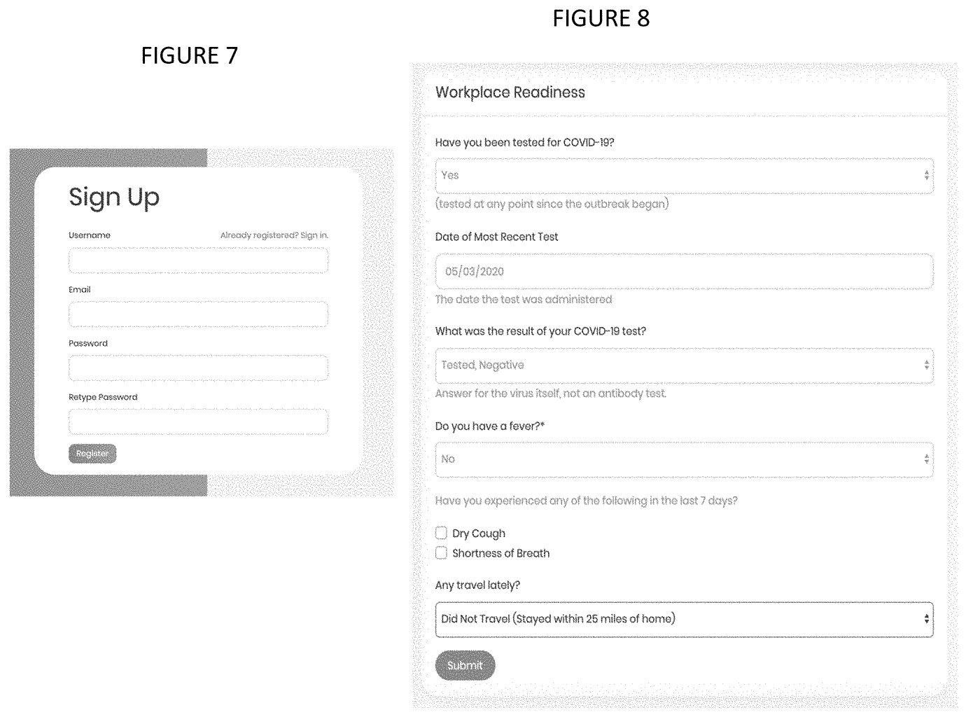

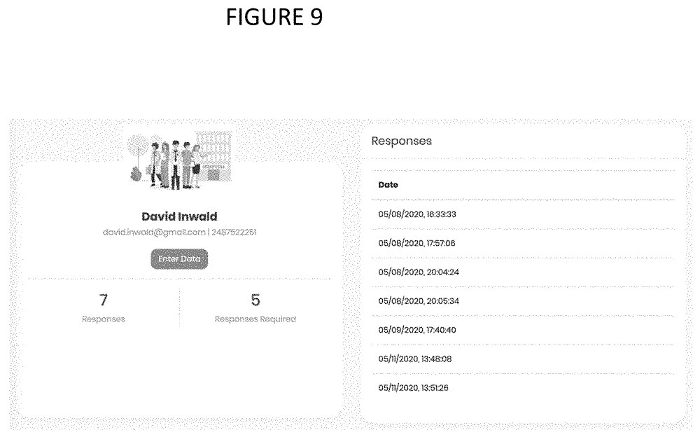

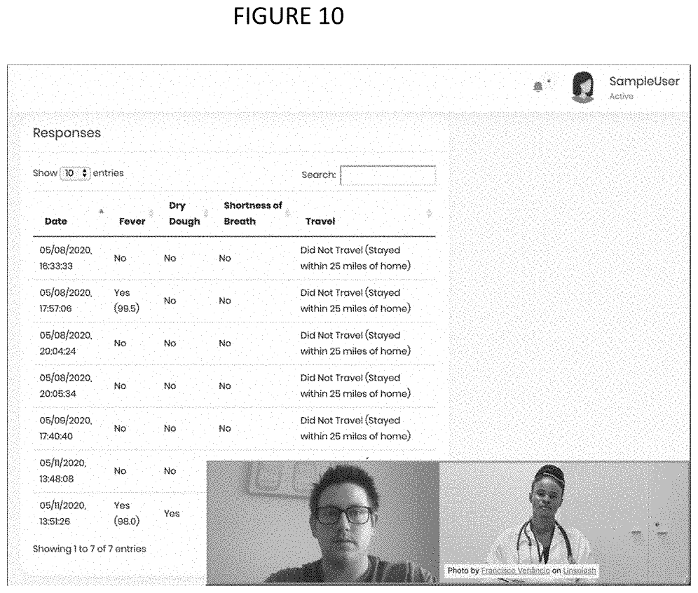

Employers/schools can manage employees/students (invitations, individual employee upload (FIG. 6A) or bulk employee/student upload (FIG. 6B) such as by uploading an Excel or CSV file to add multiple employees with one click), view dashboard summaries, view status of individual employees/students, and generate reports. The workplace dashboard, shown in FIG. 5, shows company information at a glance and employers can add new employees and download reports. Employees/students can register with the application 10 (FIG. 7), agree to terms, fill out questionnaires (as shown in FIG. 8), and optionally participate in telemedicine with health care professionals. Simple registration includes username, email, and password. Companies/schools can also require employees/students to agree to specific terms/contracts prior to entering data. An employee/student dashboard, shown in FIG. 9, can be provided to organize the information for the employee/student in a central place in the application 10. Health care professionals can review employee/student symptoms over time, and record findings or observations. Some workplaces have staff clinicians that can carry out telehealth or physical exams. When a clinician uses the application 10 for a virtual interview/exam, the result of their findings is stored in the database without directly allowing access to PHI by the employer. FIG. 10 shows an example telehealth interview.

Heads of companies, human resources employees, or company health care professionals can receive reports at any time periods, such as hourly, daily, or weekly, on the health status of employees who are using the application 10. An alert can be sent to the employers/school from the application 10 when an employee/student enters in the application 10 that they have experienced symptoms of an infectious disease. The employer/school can then decide if the employee/student should stay home if not at work/school yet, go home if at work/school, and/or remain at home if they had been sent home previously. Any criteria for decisions can be set by the employer/school, the federal government, the state government, or by local government policies. Not all workplaces require the same screening criteria. To account for this, the screening criteria is fully customizable. Options include which symptoms to include, how long an employee/student needs to be symptom-free, travel requirements, and more.

The application 10 can also provide any necessary analysis on the symptoms experienced by the employee/student and determine if it is likely that the employee/student has an infectious disease, so that the employer/school can make an informed decision on whether to send the employee/student home or allow them back to work, and the application 10 or employer/school can suggest that the individual be tested for an infectious disease. The application 10 can also notify the employer/school when sufficient time has passed since the symptoms that the employee/student is not considered infectious and can return to work.

In addition to customizing the screening criteria, the application 10 can be white-labeled for groups/organizations that want to offer a branded interface unique to their employees. Beyond branding, white-labeling enables larger organizations to store data in their own private database with particular security configurations. The application 10 can be integrated into existing employee management systems, CRMS, etc. Offered via an API, employers can use the application 10 within their native environments. The telehealth program can be compatible with the browsers Chrome 58+, Firefox 56+, Safari 11+, or Operate 45+.

Therefore, the present invention provides for a method of monitoring the health of employees or students, by an employee/student inputting data about medication, lifestyle, symptoms of infectious disease, and user defined metrics in an application, performing an analysis on the data, and outputting a result from the data tracking symptom progression, alerting an employer/school about the status of the employee's or student's symptoms, and indicating either that the employee/student should be sent home or continue to work.

Any of the other steps described above can also be included in this method, including tracking anxiety over time, tracking geolocation of symptoms and outbreaks, and tracking trends among individuals without symptoms and individuals with different diseases, and integrating data from outside databases.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### Example 1

This Example demonstrates a practical use case of the present invention for a K-12 school. The school, with approximately 150 employees and approximately 450 students initialized use of software prior to returning to in-person learning. This time period was used to provide customized symptom screening designed to meet the needs of the school—determined by healthcare professionals, regulatory guidelines, and their internal liability assessment team. Data was quickly onboarded using exported file formats from the school's School Management Software. After a staff-only period of use, the customized application was extended to student families, totaling approximately 600 individuals per day. Each day, these individuals receive email and/or SMS communications prompting the individual to enter responses for that day. On average, the questionnaires take about 45 seconds per individual to complete. The school has a designated set of staff members with access to review student information, in this case, one staff member per grade level. Building entrances were then separated, enabling each grade level to have their own isolated entrance.

### Example 2

This is continued from the example school from EXAMPLE 1. After noticing an increase in the number of failed screenings, the present invention's data analytics dashboard was able to assist in identifying two subgroups (grade levels) where the failed screenings were elevated. In reaction, the school was able to enforce a policy where the two grade levels with elevated failed screened engaged in remote learning for a period of two weeks while the rest of the school continued with in-person learning. During the off-period, the two isolated groups continued to see rises in failed screening occurrences, while the remaining group's data reflected average activity, thus naively validating the decision to isolate the group. Without the use of the present invention, the information to make a data-driven decision would be either inaccurate or labor intensive.

### Example 3

This is another continuation with the school referenced in EXAMPLE 1, whereby after a one-week “vacation” period a reviewer was able to identify an elevated number of responses to a question posed to staff and students regarding out-of-state travel. Surfacing this information in a dashboard (similar to EXAMPLE 2) enabled administrators to make the decision to enforce a week of remote learning before returning to in-person learning after the vacation period. This major decision was made easier by bringing more tangible data to the conversation, in this case, that about 15% of individuals reported leaving the state the week prior.

### Example 4

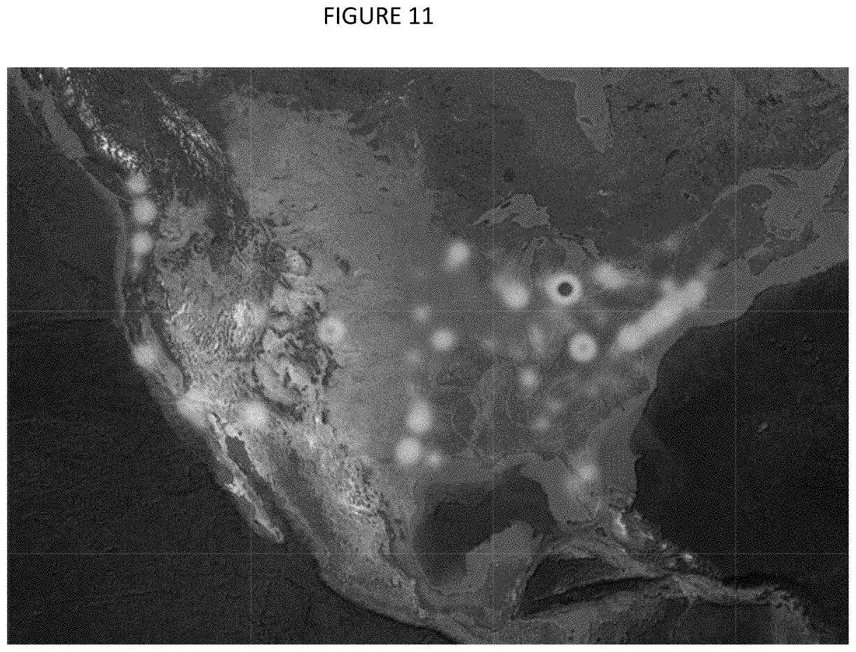

This is a heatmap demonstration of user engagement with the present invention's hosted application, shown in FIG. 11. To date (May 2021), the present invention had gathered a total of >200,000 responses across >3,000,000 data points. The data collected by the present invention can be invaluable to public health—as the detailed record maintained by the database systems represents a more thorough record than many other datasets since the onset of the COVID-19 pandemic.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. An application for tracking infectious disease, stored on non-transitory computer readable media comprising:
an input module for inputting variables from a user in electronic communication with an output variable module;
an analysis module for analyzing input variables and output variables; and
an output module for presenting results to the user.

2. The application of claim 1, wherein the disease tracked is an infectious disease chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, and zika virus.

3. The application of claim 1, wherein said input module receives data from users in a medication question module, and lifestyle question module.

4. The application of claim 1, wherein said output variable module includes a symptom question module, and user defined metrics question module.

5. The application of claim 1, wherein said input module receives data from outside devices chosen from the group consisting of general fitness trackers, heartbeat trackers, heart rate trackers, skin temperature trackers, respiratory rate trackers, body posture trackers, eyesight trackers, blood oxygen trackers, glucose level trackers, sleep trackers, body temperature trackers, skin conductance trackers, and combinations thereof.

6. The application of claim 1, wherein said input module receives data from outside databases chosen from the group consisting of clinics, electronic medical records (EMRs), pharmaceutical companies, private databases, weather monitoring systems, and CROs.

7. The application of claim 8, wherein said analysis module finds other individuals with similar data as the user to predict infection risk.

8. The application of claim 1, wherein said analysis module includes analysis methods of regressions, time series, random forest, classifiers, neural networks, support vector machines, Al/machine learning techniques, miscellaneous classical statistical techniques, and combinations thereof.

9. The application of claim 1, wherein said output module displays strongest trends, key performance indicators, and tracking over time.

10. The application of claim 1, wherein said application is in electronic communication with external databases and healthcare professionals.

11. The application of claim 1, further including an alarm for reminding the user to input data into said input module and said output variable module.

12. The application of claim 1, further including a telehealth module for conducting telehealth interviews and storing results in a database.

13. The application of claim 1, further including a dashboard that organizes information for the individual in a central place.

14. A method of tracking infectious disease, including the steps of:
a user inputting data about symptoms of infectious disease and user defined metrics in an application stored on non-transitory computer readable media;
performing an analysis on the data; and
outputting a result from the data tracking symptom progression, tracking geolocation of symptoms and outbreaks, and tracking trends among individuals without symptoms and individuals with different diseases.

15. The method of claim 14, wherein said inputting step further includes the step of integrating a user's data from outside devices chosen from the group consisting of general fitness trackers, heartbeat trackers, heart rate trackers, skin temperature trackers, respiratory rate trackers, body posture trackers, eyesight trackers, blood oxygen trackers, glucose level trackers, sleep trackers, body temperature trackers, skin conductance trackers, and combinations thereof.

16. The method of claim 14, wherein said inputting step further includes the step of integrating data from outside databases chosen from the group consisting of clinics, electronic medical records (EMRs), pharmaceutical companies, private databases, weather monitoring systems, and CROs.

17. The method of claim 14, wherein said performing an analysis step is further defined as performing an analysis method chosen from the group consisting of regressions, time series, random forest, classifiers, neural networks, support vector machines, Al/machine learning techniques, miscellaneous classical statistical techniques, and combinations thereof.

18. The method of claim 14, wherein the disease tracked is an infectious disease chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, and zika virus.

19. The method of claim 14, wherein said outputting step further includes displaying strongest trends, key performance indicators, and tracking over time.

20. A method of monitoring the health of employees or students, including the steps of:
an employee or student inputting data about symptoms of infectious disease and user defined metrics in an application stored on non-transitory computer readable media;
performing an analysis on the data;
outputting a result from the data tracking symptom progression;
alerting an employer or school about the status of the employee's or student's symptoms; and
indicating either that the employee or student should be sent home or continue to work.

21. The method of claim 20, further including the steps of establishing screening criteria for employees/students, creating a workplace, and adding employees/students before said inputting step.

22. The method of claim 20, further including the step of conducting a telehealth interview with the employee/student.

23. The method of claim 20, further including the step of indicating that the employee/student should be tested for infectious disease.

24. The method of claim 20, further including the steps of tracking anxiety over time, tracking geolocation of symptoms and outbreaks, and tracking trends among individuals without symptoms and individuals with different diseases, and integrating data from outside databases.

25. The method of claim 20, wherein the disease tracked is an infectious disease chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, and zika virus.
